(19) United States
(12) Patent Application Publication
(10) Pub. No.: US 2003/0018991 A1
(43) Pub. Date: Jan. 23, 2003

(54) Transformation and regeneration of wheat using increased copper levels

(76) Inventors: Paul A. Lazzeri, Tarragona (ES); Sonriza Rasco-Gaunt, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/178,433

(22) Filed: Jun. 24, 2002

Related U.S. Application Data
(60) Provisional application No. 60/302,094, filed on Jun. 29, 2001.

(51) Int. Cl. A01H 5/00; A01N 43/60; A01N 43/56; A01N 43/647; A01N 43/713

(52) U.S. Cl. 800/278; 504/145; 504/136; 504/139

(57) ABSTRACT

A regeneration medium for use in wheat transformation which utilises copper at a concentration in a range from greater than 50 μM to less than or equal to 300 μM and a growth hormone at a concentration in a range from 0.05 to 10 mg l⁻¹ are disclosed. Growth hormone is added to the regeneration medium if wheat tissue or cell has not been treated with a growth hormone after callus induction and prior to using the wheat regeneration medium. A method for producing a transformed wheat plant using this regeneration medium is also disclosed.

This application claims the priority benefit of U.S. Provisional Application 60/302,094, filed Jun. 29, 2001, the disclosure of which is hereby incorporated by reference in its entirety.

The present invention concerns the transformation and regeneration of plants, in particular, wheat using increased copper levels.

More than 50% of the food used by man is provided by a single group of plants, the cereals, of which wheat is one of the most important species.

Introduction of genes using genetic engineering is dependent on efficient, reproducible in vitro culturing methods. Virtually all current genetic engineering technologies require that genes be delivered to cells grown in vitro. Most published methods for generating fertile transformed plants from cereals (e.g., rice, wheat, maize, oat, sorghum, triticale, barley and rye) use explants taken from immature tissues of the donor plant for culture establishment. These immature tissues are frequently still undergoing cell division and show a high capacity for proliferation under tissue culture conditions. Among the most popular explants for culture initiation are immature embryos or the scutellum of the embryo (the portion remaining after removal of the embryo axis), immature inflorescences, shoot meristems, immature leaf tissues or microspores (immature pollen grains). From these initial explants, cellular proliferation occurs to produce a callus tissue. Gene delivery (for transformation) may be effected immediately after culture initiation, or after a period of proliferation. After gene delivery, targeted explants are grown on to produce regenerable cultures from which plants may be regenerated. To distinguish transgenic tissues from non-transformed tissues, an in vitro selection system is most commonly used. This typically involves the expression of a selectable marker gene in transgenic cells, which confers resistance to the corresponding toxic selection agent. Among the selection agents which may be used are antibiotic molecules, herbicidal compounds or other cytotoxic compounds. Selection pressure may be applied during culture proliferation, during regeneration or during both culture phases.

The development of an efficient transformation system is necessary for the molecular analysis of gene expression in plants and for commercial application of transformation technology. In cereal crop plants, this development has been slowed by difficulties encountered in plant regeneration and the relative inefficiency of Agrobacterium-mediated transformation in monocots. Most of the progress that has been made in the transformation of cereals has been in producing transgenic rice and maize. Progress in wheat has been hampered by the inability to establish suitably efficient techniques for the regeneration of fertile plants following transformation. This problem has been particularly acute in elite genotypes (current agronomically or genetically important germplasm).

Plant tissue culture media used for the regeneration of cereals typically include inorganic macro and micronutrients, some essential organic supplements such as vitamins and inositol, a carbon source and plant growth regulators. Inorganic macronutrient and micronutrient levels used in most plant tissue culture media are based on levels established in a medium developed by Murashige & Skoog, Physiol. Plant. 15: 473-497 (1962) for tobacco tissue culture. For many of the micronutrients, however, no clear optimal level was apparent. These micronutrient levels which were adequate for tobacco tissue culture and regeneration may not be optimum for culture of other plant species such as the cereal monocots.

Recent studies in wheat (Triticum aestivum L.) have shown that an increased level of the micronutrient copper in the culture medium dramatically improves regeneration. One group examined the effects of six copper levels on regeneration from callus cultures in hexaploid wheat (Purnhauser, Cereal Res. Comm. 19: 419-423 (1991)). It was found that regeneration rates were eight times higher on medium containing 10 μM CuSO₄ than on the original MS copper level (0.1 μM). A few years later similar results were found using wheat anther cultures and triticale immature embryo-derived callus (Purnhauser et al., Plant Cell Tiss. Org. Cult. 35: 131-139 (1993)). Still another group tested the effects of different copper levels on the production of embryoids from cultures of the anther of tetraploid (pasta) wheat (T. turgidum L.). In this experiment, four wheat genotypes were tested. In three of the four, the addition of 20 or 40 μM CuSO₄ to the culture medium increased the production of embryoid from anthers by comparison with control treatment, but there was no significant difference between the 20 and 40 μM CuSO₄ treatments. In the fourth genotype the addition of supplementary CuSO₄ reduced embryoid production at all levels tested (10, 20 or 40 μM) (Ghaemi et al., Plant Cell Tiss. Org. Cult. 36: 355-359 (1994)).

These studies demonstrated that the addition of copper to wheat culture media could stimulate regeneration. However, the procedures described did not result in the type of high-frequency regeneration system applicable in elite germplasm that is required for efficient wheat transformation. In the Purnhauser et al. 1991 study, optimum regeneration was seen with 10 μM Cu (although “more detailed” studies which gave an optimum Cu level of 2 μM were cited), but the maximum regeneration frequency achieved was 59% and the study was made with a single breeding line (not a variety) which had previously been selected for good response in culture. In the later study on the effect of copper on embryoid production in tetraploid wheat (Ghaemi et al., 1994), while three of the four genotypes did show increased embryoid production at either 20 or 40 μM, in the fourth genotype supplementary Cu had significant negative effects on embryogenesis and over the four genotypes the production of green plants from copper-containing media was actually lower than from control media.

In contrast, the present invention demonstrates that an improved, high-copper medium allows reliable high-frequency regeneration from a range of current elite wheat germplasm leading to up to 12-fold increases in the production of fertile transgenic plants. This provides a broadly-applicable, efficient and reliable procedure for wheat genetic modification.

### SUMMARY OF THE INVENTION

This invention concerns a regeneration medium for use in wheat transformation comprising copper at a concentration in a range from greater than 50 μM to less than or equal to 300 μM and growth hormones at concentrations in a range from 0.05 to 10 mg l⁻¹, provided that the growth hormone is added to the regeneration medium if wheat tissue or cell has not been treated with a growth hormone after callus induction and prior to using the wheat regeneration medium.

In another embodiment, this invention concerns a method for producing a transformed plant which comprises:

a) transforming a regenerable tissue or cell from wheat with a recombinant DNA construct to produce transformed regenerable tissue or cell comprising the recombinant DNA construct;

b) culturing the transformed tissue or cell on regeneration medium comprising copper at a concentration in a range from greater than 50 μM to less than or equal to 300 μM and a growth hormone at a concentration in a range from 0.05 to 10 mg l⁻¹ provided that the growth hormone is added to the regeneration medium if wheat tissue or cell has not been treated with a growth hormone after callus induction and prior to using the wheat regeneration medium; and

c) regenerating a transformed wheat plant from the transformed tissue or cell.

### BRIEF DESCRIPTION OF THE FIGURES

FIG. 1 shows the effect of copper levels from 100 to 1000 μM on wheat in vitro regeneration, illustrating particularly the effect on shoot quality. Two wheat genotypes were used for the experiment: Riband (top row) and Imp (bottom row). The various treatments are compared with the control shoots grown in 0.1 μM copper.

FIG. 2a compares the plant regeneration frequencies of the two wheat genotypes Riband and Imp treated with different copper levels. Copper was supplemented up to 1000 μM.

FIG. 2b shows the effect of copper levels from 0.1 (control) to 1000 μM on the quantity of plants regenerated by two wheat varieties Eole and NH535.

FIG. 3a shows the effect of 100 μM copper treatment on recovery of transgenic wheat lines from three different selection systems (bar or mopat) using variety Cadenza.

FIG. 3b shows the effect of 100 μM copper treatment on frequency of plant escapes from three different selection systems (bar or mopat) using variety Cadenza.

FIG. 3c shows the effect of 100 μM copper treatment on recovery of transgenic wheat lines from PPT (pHP8092) or sulfonylurea (pML146) selection systems using variety NH535.

FIG. 3d shows the effect of 100 μM copper treatment on frequency of plant escapes from PPT (pHP8092) or sulfonylurea (pML146) selection systems using variety NH535.

FIG. 4a shows the effect of 100 μM copper treatment on recovery of transgenic lines from different wheat genotypes using PPT selection.

FIG. 4b shows the effect of 100 μM copper treatment on frequency of plant escapes from different genotypes using PPT selection.

### DETAILED DESCRIPTION OF THE INVENTION

All patents, patent applications and publications referred to herein are incorporated by reference in their entirety.

In the context of this disclosure, a number of terms shall be utilized.

As used herein, “Regeneration medium” (RM) promotes differentiation of totipotent embryogenic plant tissues into shoots, roots and other organized structures and eventually into plantlets that can be transferred to soil. “Plant culture medium” is any medium used in the art for supporting viability and growth of a plant cell or tissue, or for growth of whole plant specimens. Such media commonly include, but are not limited to, macronutrient compounds providing nutritional sources of nitrogen, phosphorus, potassium, sulfur, calcium, magnesium, and iron; micronutrients, such as boron, molybdenum, manganese, cobalt, zinc, copper, chlorine, and iodine; carbohydrates; vitamins; phytohormones; selection agents; and may include undefined components, including, but not limited to, casein hydrolysate, yeast extract, and activated charcoal. The medium may be either solid or liquid.

“Plant cell” is the structural and physiological unit of plants, consisting of a protoplast and the cell wall.

“Plant tissue” is a group of plant cells organized into a structural and functional unit.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transferred nucleic acid fragments are referred to as “transgenic” or “transformed” organisms. The preferred method of cell transformation of rice, corn and other monocots is the use of particle-accelerated or “gene gun” transformation technology (Klein et al, (1987) Nature (London) 327: 70-73; U.S. Pat. No. 4,945,050), or an Agrobacterium-mediated method using an appropriate Ti plasmid containing the transgene (Ishida Y. et al, 1996, Nature Biotech. 14: 745-750).

The term “isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques. A “recombinant DNA construct” comprises an isolated polynucleotide operably linked to at least one regulatory sequence. The term also embraces an isolated polynucleotide comprising a region encoding all or part of a functional RNA and at least one of the naturally occurring regulatory sequences directing expression in the source (e.g., organism) from which the polynucleotide was isolated, such as, but not limited to, an isolated polynucleotide comprising a nucleotide sequence encoding a herbicide resistant target gene and the corresponding promoter and 3′ end sequences directing expression in the source from which sequences were isolated.

A “transgene” is a recombinant DNA construct that has been introduced into the genome by a transformation procedure.

“PCR” or “Polymerase Chain Reaction” is a technique for the synthesis of large quantities of specific DNA segments, consists of a series of repetitive cycles (Perkin Elmer Cetus Instruments, Norwalk, CT). Typically, the double stranded DNA is heat denatured, the two primers complementary to the 3′ boundaries of the target segment are annealed at low temperature and then extended at an intermediate temperature. One set of these three consecutive steps is referred to as a cycle.

“Selection agent” refers to a compound toxic to non-transformed plant cells which kills non-transformed tissues when it is incorporated in the culture medium. Cells can be transformed with an appropriate gene, such that expression of that transgene confers resistance to the corresponding selection agent, via detoxification or another mechanism, so that these cells continue to grow and are subsequently able to regenerate plants. The gene conferring resistance to the selection agent is termed the “selectable marker gene”, “selectable marker” or “resistance gene”. Transgenic cells that lack a functional selectable marker gene will be killed by the selection agent. Selectable marker genes include genes encoding antibiotic resistance, such as those encoding neomycin phosphotransferase II (neo or nptII) and hygromycin phosphotransferase (hpt) as well as genes conferring resistance to herbicidal compounds. Herbicide resistance genes generally code for a modified target protein insensitive to the herbicide or for an enzyme that degrades or detoxifies the herbicide in the plant before it can act (DeBlock et al., 1987, EMBO J. 6: 2513-2518, DeBlock et al., 1989, Plant Physiol., 91: 691-704). For example, resistance to glyphosate or sulfonylurea herbicides has been obtained by using genes coding for mutant versions of the target enzymes, 5-enolpyruvylshikimate-3-phosphate synthase (EPSPS) and acetolactate synthase (ALS). Resistance to glufosinate ammonium, bromoxynil and 2,4-dichlorophenoxyacetic acid (2,4-D) has been obtained by using bacterial genes encoding phosphinothricin acetyltransferase, a nitrilase, or a 2,4-dichlorophenoxyacetate monooxygenase, which detoxify the respective herbicide. “Sulfonylurea herbicides” include but are not limited to Rimsulfuron, Nicosulfuron, Classic, and Oust. A specific selection agent may have one or more corresponding selectable marker genes. Likewise, a specific selectable marker gene may have one or more corresponding selection agents. It is appreciated by one skilled in the art that a selection agent may not be toxic to all plant species or to all cell types within a given plant. For a plant species susceptible to a given selection agent, it is also appreciated that resistance cells, tissues or whole plants may be obtained independent of the transformation process, e.g., through chemical mutagenesis of the target gene or gene amplification of the target gene during tissue culture.

The present invention concerns a regeneration medium for use in wheat transformation comprising copper at a concentration in a range from greater than 50 μM to less than or equal to 300 μM and a growth hormone at a concentration in a range from 0.05 to 10 mg l⁻¹ provided that the growth hormone is added to the regeneration medium if wheat tissue or cell has not been treated with a growth hormone after callus induction and prior to using the copper-containing wheat regeneration medium.

More preferably, the copper concentration is in a range from 100 μM to 200 μM.

While it is possible to regenerate plants from wheat tissue cultures in the absence of growth hormones, in most genotypes, and especially elite germplasm, regeneration is stimulated by the addition of growth hormones to the medium. The growth hormones most commonly used for this purpose are natural or synthetic auxins and natural or synthetic cytokinins, although other classes of growth hormone, such as abscisic acid or giberellins may also be beneficial in some wheat genotypes. It is common for a combination of hormones, typically including an auxin and a cytokinin to be used.

Members of the above-mentioned classes of growth hormone at concentrations in a range from 0.05 to 10 mg l⁻¹ can be used to practice the invention. Those skilled in the art will appreciate that growth hormone is added to the regeneration medium if wheat tissue or cell has not been treated with a growth hormone after callus induction and prior to using the high-copper wheat regeneration medium

Examples of growth hormones that can be used to practice the invention include, but are not limited to, natural or synthetic auxins, natural or synthetic cytokinins, dicamba, abscisic acid, giberellins and chlorocholine chloride. “Dicamba” and “3,6-dichloro-o-anisic acid” are used interchangeably herein.

Examples of natural auxins include, but are not limited to indole acetic acid (IAA) and napthaleneacetic acid (NM).

Examples of synthetic auxins include, but are not limited to, monochlorophenoxyacetic acid (MCPA), dichlorophenoxyacetic acid (2,4-D), trichlorophenoxyacetic acid (2,4,5-T), 4-amino-3,5,6-trichloropicolinic acid (Picloram) and indole-3-butryic acid (IBA). Examples of natural cytokinins include, but are not limited to (6-[4-hydroxy-3-methylbut-2-enylamino]purine) (Zeatin) and 6-furfurylaminopurine (Kinetin).

“Gibberellins” and “gibberellic acid” are used interchangeably herein.

Examples of synthetic cytokinins include, but are not limited to 6-Benzylaminopurine (BAP) and 1-phenyl-3-(1,2,3-thiadiazol-5-yl)urea (Thiadiazuron).

The regeneration medium can further comprise a selection agent.

Examples of suitable selection agents, include but are not limited to, antibiotics like kanamycin, geneticin (also known as G418), paromomycin, cytotoxic agents such as hygromycin, sulfonylurea herbicides such as Nicosulfuron and Rimsulfuron, and other herbicides which act by inhibition of the enzyme acetolactate synthase (ALS), glyphosate, bialaphos and phosphinothricin (PPT). It is also possible to use positive selection marker systems such as phospho-mannose isomerase and similar systems which confer positive growth advantage to transgenic cells.

Furthermore, the regeneration medium of the invention may also comprise silver nitrate at a concentration in the range from 1 to 20 mg l⁻¹. As those skilled in the art will appreciate, the presence of silver nitrate is optional depending upon the tissue culture response of the particular wheat genotype being cultured. It is particularly useful in the case of elite germplasm showing poor response in culture where it promotes culture growth and regeneration. Other possible components that may be added to the regeneration medium include, but are not limited to, macro- and microsalts, vitamins, myo-inositol, and 3 to 9% w/v maltose.

The present invention can be used with any type of wheat. Wheat plants are classified by habitat, functional characteristics of the grain and genetic make-up; the bread wheats are hexaploid and classified as winter or spring based on habitat requirement, hard or soft based on grain function, and red or white based on seed coat color, the durum wheats are tetraploids, and the diploid wheats such as Triticum monococcum have also been grown as crops in the past. Spring wheats do not require vernalization (low temperature treatment) while winter wheats may or may not require. Examples of spring wheat include, but are not limited to, Avans (Svalof, UK), Canon (Svalof, UK), Imp (Nickersons, UK). Examples of winter wheat include, but are not limited to, Brigadier (Advanta, UK), Cadenza (CPB Twyfords, UK), Eole (Hybrinova, France), Rialto (PBI, UK), Riband (PBI, UK) and Soissons (Desprez, France).

Any regenerable plant tissue can be used in accordance with the present invention. Regenerable plant tissue generally refers to tissue which can be regenerated into a differentiated plant. For example, such tissues can include calluses and/or somatic embryos derived from whole zygotic embryos, isolated scutella, anthers, inflorescences and leaf and meristematic tissues.

Regenerable tissue cultures are typically established by taking donor plant tissue (e.g. immature caryopses containing embryos, immature spikes for inflorescence or anther cultures) from plants grown under controlled conditions in a glasshouse or controlled environment room. The tissues are surface-sterilized using agents such as ethanol and sodium hypochlorite solutions and then dissected under sterile conditions to allow the isolation of sterile explants such as embryos, scutella, inflorescences.

The explants are placed on solidified culture medium to induce cell proliferation and the induction of regenerable (embryogenic or organogenic) tissue cultures.

A recombinant DNA construct may be introduced into the regenerable plant tissue early in the culture process (i.e. within 0-24 hrs after plating of explants onto media), when no or little cell proliferation has occurred, or may be introduced at a later stage when cell proliferation has produced new callus tissue. Any method can be used to transform regenerable plant tissue. There can be mentioned particle bombardment, Agrobacterium transformation, electroporation of regenerable tissues (D'Halluin, K., Bonne, E., Bossut, M., De Beuckleer, M. and Leemans, J. (1992) Plant Cell 4, 1495-1505), the use of silicon carbide whiskers (Wang, K., Drayton, P., Frame, B., Dunwell, J. and Thompson, J. (1995) In Vitro Cell and Developmental Biology 31, 101-104) and protoplast-facilitated gene delivery Rhodes, C. A., Pierce, D. A., Mettler, I. J., Mascarenhas, D. and Detmer, J. J. (1988) Science 240, 204-207. The preferred method for transformation is the particle bombardment method.

For cereals, the media used for the induction phase of tissue culture typically contain auxins such as phenoxyacetic acids (particularly 2,4-D), Picloram, NAA and IBA or Dicamba, typically at concentrations in the range (0.1-4.0 mg/l) which may or may not be combined with other growth regulators such as cytokinins or abscisic acid.

Callus induction takes place over a period of some one to five weeks, at which time a regenerable tissue will have developed from the initial explant and typically embryogenic cells or somatic embryos will be present. The induction culture may be performed in darkness or under light depending on the species and genotype concerned.

In order to identify transformed tissues, cultures may be exposed to a selection agent appropriate to a selectable marker gene included in the recombinant DNA construct used for transformation. The selection agent may be supplied during the callus induction or proliferation phases of culture, or may be supplied during culture on regeneration medium. Single, or more commonly multiple passages of selection may be applied. Even when a resistance gene is expressed in transformed tissues it is common for the application of selection to reduce the efficiency of formation of regenerable tissue from transformed cells (e.g. to reduce the frequency of somatic embryogenesis). Thus, it is preferable to supply the selection agent during the regeneration phase of culture rather than during the induction phase in order to increase the efficiency of formation of regenerable tissue from transformed cells.

To achieve plant regeneration from cereal cultures, after around four weeks of culture on induction medium regenerable plant tissue is placed in a medium capable of stimulating shoot formation and growth. Shoot formation may be completed after a single passage (typically three to four weeks) on regeneration medium, but it is common for multiple passages on regeneration medium to be used.

When shoots are sufficiently large to be picked individually from the parent tissue culture they are usually transferred to a low-salt and growth-hormone free medium for one or more passages to allow good root development and further shoot growth before subsequent transfer to soil. Immediately after transfer to soil plantlets are maintained under high humidity for several days to allow acclimatization, and may then be grown on in the same way as seed-grown plants.

Culture media used for regeneration have similar basic composition to those used for callus induction, but differ in that they typically contain lower levels of auxin and that they most commonly contain cytokinin. Other plant growth regulators may also be present. The regeneration medium of the present invention comprises (a) copper at a concentration in a range from greater than 50 μM to less than 300 μM and (b) a growth hormone at a concentration in a range from 0.05 to 10 mg l⁻¹ provided that the growth hormone is added to the regeneration medium if wheat tissue or cell has not been treated with a growth hormone after callus induction and prior to using the wheat regeneration medium.

The use of the improved regeneration medium containing high levels of copper offers a number of significant advantages over standard media.

Transformation efficiency in the high-copper medium is improved. By improved efficiency, it is intended that the number of transformed plants recovered by a transformation attempt is increased preferably at least two-fold, preferably at least three-fold, and more preferably at least twelve-fold. Shoots recovered from the improved medium are of higher quality, being larger and more vigorous, with reduced leaf curling. They can be separated from the parent culture more efficiently and with less damage so that effectively more plants are produced and their improved vigor means that fewer plants are lost to the stresses associated with transfer from in vitro conditions to the glasshouse.

A significant observation is that on the improved high-copper medium elite wheat cultivars show high efficiency regeneration similar to that normally seen only from tissue culture model genotypes which have been specifically selected as amenable in culture. This is reflected in the fact that, on the improved medium, transformation efficiencies from elite varieties can equal or exceed levels previously seen only in model genotypes.

High-copper medium has a synergistic effect with selection procedures as it facilitates selection at the regeneration stage, as distinguishing transgenic (resistant) shoots from escapes is more efficient in vigorous fast-growing cultures than in cultures where both escapes and transgenics have slow growth rates.

In another embodiment, this invention concerns a method for producing a transformed plant which comprises:

a) transforming a regenerable tissue or cell from wheat with a recombinant DNA construct to produce transformed regenerable tissue or cell comprising the recombinant DNA construct;

b) culturing the transformed tissue or cell on regeneration medium comprising copper at a concentration in a range from greater than 50 μM to less than 300 μM and a growth hormone at a concentration in a range from 0.05 to 10 mg l⁻¹ provided that the growth hormone is added to the regeneration medium if wheat tissue or cell has not been treated with a growth hormone after callus induction and prior to using the wheat regeneration medium; and

c) regenerating a transformed wheat plant from the transformed tissue or cell.

Transformation of regenerable tissue and subsequent culturing on the regeneration medium of the invention are discussed above.

The disclosure of each reference set forth herein is incorporated herein by reference in its entirety.

The following examples are offered by way of illustration and not by way of limitation.

### EXAMPLE 1

### Wheat Transformation Methodology

A. Preparation of Plant Materials

Winter and spring genotypes were used for the various experiments described in the Examples below, namely: winter wheats BO014 (Hybrinova, France), Cadenza (CPB Twyfords, United Kingdom), DZ544 (Hybrinova, France), Eole (Hybrinova, France), NH535 (Hybrinova, France) and Riband (PBI, United Kingdom), and spring wheat Imp (Nickersons, United Kingdom). These genotypes are all current commercial varieties or elite breeding lines used as parents in current hybrid wheat varieties. Winter wheat seeds except genotype Cadenza were sown in plug trays containing a commercial soil mix (Petersfield Products, United Kingdom) with the following composition: 75% L&P fine-grade peat, 12% screened sterilised loam, 10% 6 mm screened, lime-free grit, 3% medium grade vermiculite, 3.5 kg Osmocote per m³ soil (slow-release fertiliser, 15-11-13 Nitrogen-Phosphorus-Potassium plus micronutrients), 0.5 kg PG mix per m³ (14-16-18 Nitrogen-Phosphorus-Potassium granular fertiliser plus micronutrients). After one week, seedlings were subjected to vernalisation at 6° C. for eight weeks. Thereafter, the seedlings were transferred in 8″ pots and grown in a controlled environment room with a 16 h photoperiod (400W HQI lamps providing irradiance of ca. 750 μE s⁻¹ m⁻²), with an 18 to 20° C. day and 14 to 16° C. night temperature, 50 to 70% relative air humidity. Cadenza (winter wheat with no vernalisation requirement) and the spring wheat Imp were directly sown in 8″ pots and grown under conditions described above. Plants were fertilized every two weeks with a 4-1-2 liquid fertilizer (Vitax Ltd, United Kingdom). Pest control was effected by the biological control of thrips using Amblyseius caliginosus (Novartis BCM Ltd, United Kingdom).

B. Isolation of Explants and Culture Initiation

The primary culture explants were immature embryos or scutella. Wheat caryopses containing early-medium milk-stage grains with translucent embryos were harvested and surface-sterilised in 70% ethanol for 5 min and 0.5% hypochlorite solution for 15 min. Under aseptic conditions, embryos of approximately 0.5-1.0 mm length were isolated and either cultured entirely or the embryo axes were removed to produce isolated scutella. Thirty embryos/scutella were placed in the center (18 mm target circle) of a 90 mm Petri dish containing callus induction medium. The callus induction medium for embryos/scutellar tissues, called MD0.5, consisted of solidified (0.5% Agargel, Sigma A3301) modified MS medium supplemented with 9% sucrose, 10 mg l⁻¹ AgNO₃ and 0.5 mg l⁻¹ 2,4-D (Rasco-Gaunt et al., 2001 J. Exp. Bot. 52 (357): 865-874). Embryos/scutella were placed with the embryo-axis side in contact with the medium. Cultures were incubated at 25±1° C. in darkness for approximately 24 hr before bombardment.

C. DNA Precipitation Procedure and Particle Bombardment

Sub-micron gold particles (0.6 μm Micron Gold, Bio-Rad) were coated with plasmid DNA following the protocol modified from the original Bio-Rad procedure (Rasco-Gaunt et al., 1999 Plant Cell Rep. 19: 118-127). The standard DNA/gold precipitation mixture consisted of 1 mg of gold particles in 50 μl sterile distilled water, 50 μl of 2.5 M calcium chloride, 20 μl of 100 mM spermidine free base and 5-10 μl DNA (concentration 1 μg μl⁻¹). After combining the components, the mixture was vortexed and the supernatant discarded. The particles were then washed with 150 μl absolute ethanol and finally re-suspended in 85 μl absolute ethanol. The DNA/gold ethanol solution was kept on ice to minimize ethanol evaporation. For each bombardment, 5 μl of DNA/gold ethanol solution (ca. 60 μg gold) was loaded onto the macrocarrier.

Particle bombardment were carried out using a DuPont PDS 1000/He particle gun with a gap distance of 2.5 cm, target distance of 5.5 cm from the stopping plate at 650 or 900 psi acceleration pressure and 28 in. Hg chamber vacuum pressure (Rasco-Gaunt et al., 1999 Plant Cell Rep. 19: 118-127).

D. Callus Induction, Selection and Regeneration of Transformants

Following bombardment, explants were distributed over the surface of MD0.5 callus induction medium in the original dish and two other dishes and cultured at 25±1° C. in darkness for three to four weeks, for the induction of embryogenic callus.

Embryogenic calluses were transferred, for regeneration, to RZ regeneration medium and cultured under 12 h light (250 μE s⁻¹ m⁻², from cool white fluorescent tubes) at 25+1° C. for three weeks. The RZ regeneration medium, contained L salts, vitamins and myo-inositol, 3% w/v maltose, 0.1 mg l⁻¹ 2,4-D and 5 mg l⁻¹ zeatin (Rasco-Gaunt and Barcelo, 1999 Plant Cell Culture Protocols. Methods in Molecular Biology series. Hall, RD ed. 111: 71-81, Humana Press, Totowa NJ) and CuSO₄ at concentrations ranging from 0 to 1000 μM. The supplementation of silver nitrate in the range of 1-20 mg l⁻¹ is optional. Selection agents such as 2-6 mg l⁻¹ Glufosinate ammonium (Greyhound, United Kingdom) and 10-500 ppm sulfonylurea herbicide e.g. Nicosulfuron (DuPont, France) and Rimsulfuron (DuPont, France) were incorporated in RZ regeneration medium. A second three-week culture passage on the same medium was performed as required, with or without a selection agent. Following culture on RZ regeneration medium, plantlets were cultured for one or two three-week passages on R0 medium (RZ without hormones, AgNO₃ and CuSO₄ supplementation), with or without the selection agent.

Rooted plantlets were transferred from R0 medium to soil and were acclimatised in a propagator for 1-2 weeks. Thereafter, the plants were grown to maturity under the growth conditions described above (“Preparation of Plant Materials”).

E. DNA Isolation from Callus and Leaf Tissues

Genomic DNA was extracted from leaves using a GenElute™ Plant Genomic DNA kit (Sigma G2N350). For quantification of genomic DNA, gel electrophoresis was performed using an 0.8% agarose gel in 1×TBE buffer. The samples (one microlitre each) were fractionated alongside 200, 400, 600 and 800 ng μl⁻¹ uncut λ DNA markers.

F. Polymerase Chain Reaction (PCR) Analysis

The presence of the transgene was analysed by PCR using Sigma (P4600) ReadyMix™ Taq with MgCl₂ using 100-200 ng genomic DNA in a 25 ml reaction. The mixture contains 20 mM Tris-HCl (pH 8.3), 100 mM KCl, 3 mM MgCl₂, 0.002% gelatin, 0.4 mM dNTP and 60 U/ml Taq DNA polymerase. Primers were identified using a Vector NTI molecular biology software.

### EXAMPLE 2

### Identification of Copper Levels for Enhancing In Vitro Regeneration

To determine the effects of increased copper concentration on shoot regeneration, embryogenic calluses were subcultured onto RZ regeneration medium (see Example 1) supplemented with CuSO₄ at concentrations ranging from 0 to 1000 μM. Prior to transfer onto RZ regeneration medium, calluses were induced from scutellar tissues. Procedures for the growth of donor plant materials, isolation of explants and culture initiation are described in Example 1. Following culture on CuSO₄ medium, the calluses were transferred to R0 medium (see Example 1). After three weeks, regeneration frequencies of calluses, average number of plant per callus, as well as shoot quality were noted.

Regeneration frequency is calculated as the number of shoot-producing calluses divided by the total number of scutella isolated. Percent (%) regeneration is transformation frequency multiplied by 100.

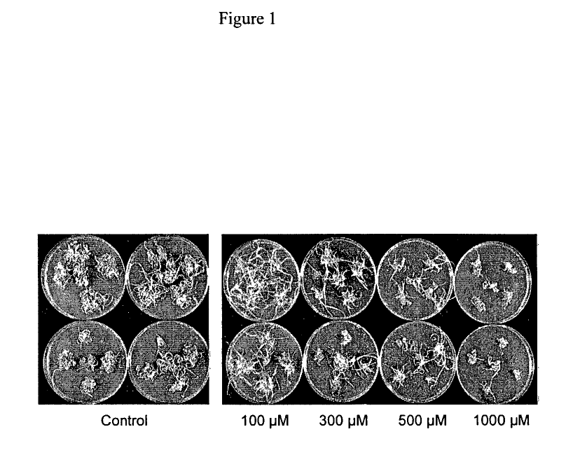

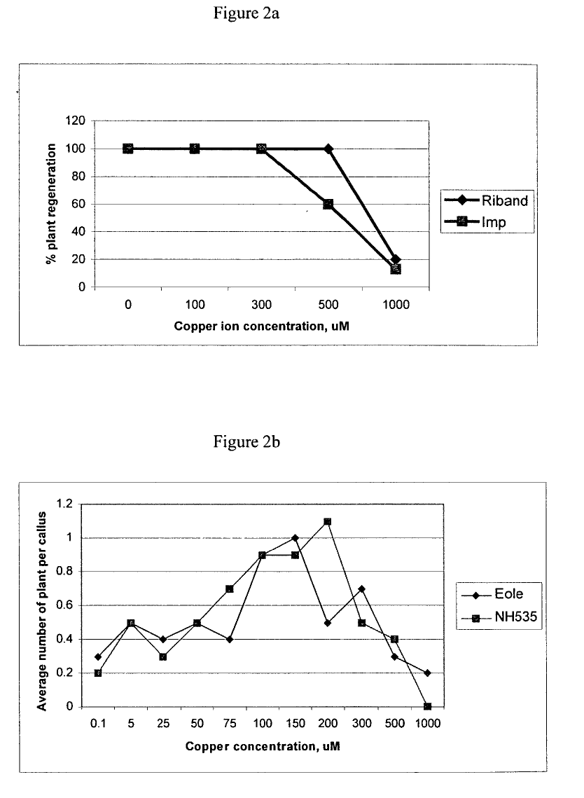

The addition of copper resulted in a strong increase in the number of regenerated plants and shoot quality. Shoots regenerated from copper levels up to 500 μM were vigorous and prolific with individual shoots being readily separable, and plants were at advanced stages of development (FIG. 1). The control cultures characteristically produced a mass of intertwined shoots and leaf structures which were hard to separate and from which only a limited number of plants could be regenerated. In improving shoot quality, copper had no negative effect on regeneration frequency. Plant regeneration frequency was at 100% in wheat variety Imp (Nickersons, United Kingdom) at CuSO₄ levels up to 300 μM and in wheat variety Riband (Plant Breeding Institute, United Kingdom) at levels up to 500 μM CuSO₄ (FIG. 2a). Similarly, in wheat varieties Eole and NH535 (Hybrinova, France), regeneration frequencies were at 100% at CuSO₄ levels up to 300 μM. FIG. 2b shows that copper also had positive effects on the number of plants regenerated per callus from these two varieties. Copper concentrations in the range of 5 to 500 μM improved regeneration. The optimum range is 100-150 μM for variety Eole, and 100-200 μM for variety NH535.

### EXAMPLE 3

### Effect of a High Copper Regeneration Medium on Transformation in a Variety of Selection Systems

To determine the overall effect of increased copper level on transformation, four different selectable marker genes or selection systems were tested. The following marker gene constructs were bombarded into scutellar tissues of wheat lines Cadenza (CPB Twyfords, United Kingdom) and NH 535 (Hybrinova, France): pHP15629 (H2B:mopat:pinII), pHP8092 (Ubi:mopat:35S), pAHC20 (Ubi:bar:nos), pML146 (ALS:Hra:ALS). Plasmids pHP15629 and pHP8092 both contain the monocot-optimised pat gene (Jayne, S., et al., U.S. Pat. No. 6,096,947) coding for resistance to phosphinothricin (PPT) driven by the maize H2B (Rice, D., WO 99/43797) or the maize Ubi-1 promoter (Christensen and Quail, 1996, Transgenic Res. 5: 213-218), respectively. The plasmid pAHC20 (Christensen and Quail, 1996, Transgenic Res. 5: 213-218) contains the bar gene, also coding for resistance to PPT, under the control of the Ubi-1 promoter. Plasmid pML146 contains a maize Hra gene encoding a mutant acetolactate synthase gene driven by its native promoter. The gene confers resistance to sulfonylurea herbicides. The transformation method used is described in Rasco-Gaunt et al. (Rasco-Gaunt et al., 1999 Plant Cell Rep. 19: 118-127, Rasco-Gaunt et al., 2001 J. Exp. Bot. 52 (357): 865-874) with modifications as cited in Example 1. Cultures or tissues bombarded with the mopat and bar constructs were selected in 4 mg l⁻¹ glufosinate ammonium during regeneration. Calluses bombarded with the Hra construct (pML146) were selected in 500 ppm Nicosulfuron during regeneration. The frequency of transformation from copper and non-copper treated cultures was determined. Transformation frequency, in this Example as in the other Examples, was calculated as the number of calluses producing transgenic plants (as confirmed by PCR analysis) divided by the total number of scutella isolated and used for bombardment. Percent (%) transformation is transformation frequency multiplied by 100. Plant escape frequency, in this Example as in the other Examples, was calculated as the number of plants that do not carry the transgene (as determined by PCR analysis) divided by the total number of putative transgenic plants regenerated from callus analysed. Percent (%) plant escape is plant escape frequency multiplied by 100.

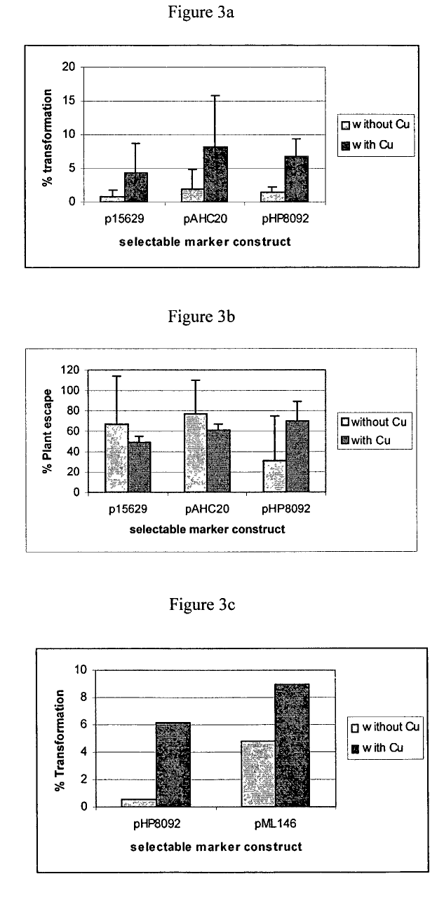

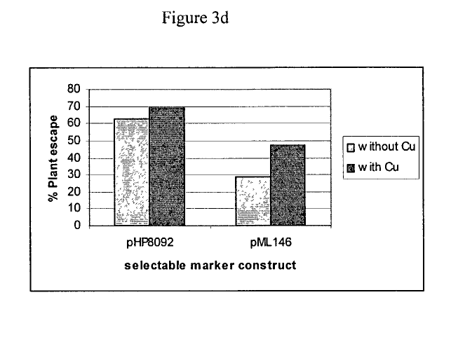

The data from two wheat genotypes shown in FIGS. 3a and 3c indicated that 100 μM CuSO₄ treatment improved transformation frequency in both PPT and sulfonylurea selection systems. In variety Cadenza (FIG. 3b), PPT selection with constructs p15629 and pAHC20 combined with copper treatment lead to a clear reduction in plant escapes. With plasmid pHP8092, PPT selection with copper treatment resulted in a slightly higher plant escape frequency in both varieties Cadenza (FIG. 3b) and NH535 (FIG. 3d). In variety NH535 (FIG. 3d), sulfonylurea selection with construct pML146 combined with copper treatment lead to a higher escape frequency.

### EXAMPLE 4

### Effect of a High Copper Regeneration Medium on Transformation of Different Wheat Genotypes

To determine the general effect of increased copper levels on transformation, five different genotypes were tested, namely: Cadenza (Cambridge Plant Breeders Twyfords, United Kingdom), BO014, DZ544, Eole and NH535 (Hybrinova, United Kingdom). The following marker and trait genes were used for transformation: pHP8092 (Ubi::mopat::35S), pWTL-Ly1 (HMW::DHDPS::HMW), pWTL-ST4 (HMW::SSI:HMW), and pCB2022 (native promoter::Pi-ta::3′UTR). Plasmid pWTL-Ly1 contains the Corynebacterium dapA lysine feedback insensitive dihydrodipicolinate synthase (DHDPS) gene under the control of the wheat High Molecular Weight (HMW) glutenin subunit promoter. For the construction of pWTL-Ly1, the HMW glutenin subunit promoter (Lamacchia et al., 2001, J. Exp. Bot. 52 (355): 243-250) was cloned into SphI/XbaI sites of pUC19, the HMW glutenin subunit terminator was cloned into BamHI/Xba I sites of above vector, a synthetic polylinker containing SnaBI/EcoRV/BstXI was cloned into XbaI site of above vector, and finally the Corynebacterium dapA dihydrodipicolinate synthase (DHDPS) gene (U.S. Pat. No. 5,773,691) was cloned into the EcoRV site of above vector. Plasmid pWTL-ST4 contains a wheat starch synthase I (SS I; NCBI GenBank Identifier (GI) No. 5880465; Li et al. (1999) TheorAppl Genet 98: 1208-1216) 1400 bp gene fragment under the control of the wheat HMW glutenin subunit promoter. For the construction of pWTL-ST4, the HMW glutenin subunit promoter was cloned into SphI/XbaI sites of pUC19, the HMW glutenin subunit terminator was cloned into BamHI/Xba I sites of above vector, a synthetic polylinker containing SnaBI / EcoRV/BstXI was cloned into XbaI site of above vector, and finally wheat starch synthase I-encoding gene fragment (1400 bp) was cloned into the EcoRV site of above vector. Plasmid pCB2022 (ATCC Deposit PTA-2631; U.S. Provisional Application No. 60/248,335) contains the rice Pi-ta gene, encoding rice blast disease resistance, driven by its 2.5 Kb native promoter (source: Greg Bryan, DuPont, USA).

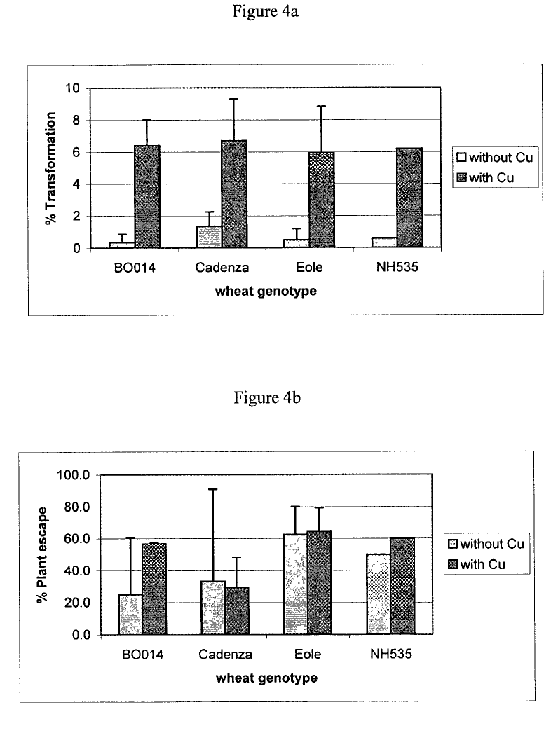

The effect of copper was examined with PPT selection and the frequency of transformation and plant escapes from copper and non-copper treated cultures was determined. The results indicate that 100 μM copper treatment improved the recovery of transformants and, transformation efficiencies were improved in all four genotypes (FIG. 4a). With PPT selection, the copper treatment did not have an important effect on the frequency of plant escapes (FIG. 4b).

What is claimed is:

1. A regeneration medium for use in wheat transformation comprising copper at a concentration in a range from greater than 50 μM to less than or equal to 300 μM and a growth hormone at a concentration in a range from 0.05 to 10 mg l⁻¹ provided that the growth hormone is added to the regeneration medium if wheat tissue or cell has not been treated with a growth hormone after callus induction and prior to using the wheat regeneration medium.

2. The regeneration medium of claim 1 wherein the copper concentration is in a range from 100 μM up to less than or equal to 200 μM

3. The regeneration medium of claim 1 or 2 which further comprises a selection agent.

4. The regeneration medium of claim 3 wherein the selection agent is selected from the group consisting of kanamycin, geneticin, paromomycin, hygromycin, glyphosate, bialaphos, phosphinothricin, and sulfonylurea herbicide.

5. The regeneration medium of claims 1 or 2 which further comprises silver nitrate at a concentration in the range from 1 to 20 mg l⁻¹.

6. The regeneration medium of claim 3 which further comprises silver nitrate at a concentration in the range from 1 to 20 mg l⁻¹.

7. The regeneration medium of claims 1 or 2 wherein the growth hormone is selected from the group consisting of 2,4-dichlorophenoxyacetic acid, indole-3-acetic acid, naphthaleneacetic acid, monochlorophenoxyacetic acid, 3,6-dichloro-o-anisic acid, 4-amino-3,5,6-trichloropicolinic acid, 6-benzylaminopurine, indole-3-butyric acid, 6-[4-hydroxy-3-methylbut-2-enylamino]purine, 6-furfurylaminopurine, 1-phenyl-3-(1,2,3-thiadiazol-5-yl)urea, abscisic acid, chlorocholine chloride and gibberellic acid.

8. The regeneration medium of claim 3 wherein the growth hormone is selected from the group consisting of 2,4-dichlorophenoxyacetic acid, indole-3-acetic acid, naphthaleneacetic acid, monochlorophenoxyacetic acid, 3,6-dichloro-o-anisic acid, 4-amino-3,5,6-trichloropicolinic acid, 6-benzylaminopurine, indole-3-butyric acid, 6-[4-hydroxy-3-methylbut-2-enylamino]purine, 6-furfurylaminopurine, 1-phenyl-3-(1,2,3-thiadiazol-5-yl)urea, abscisic acid, chlorocholine chloride and gibberellic acid.

9. A method for producing a transformed plant which comprises:
a) transforming a regenerable tissue or cell from wheat with a recombinant DNA construct to produce transformed regenerable tissue or cell comprising the recombinant DNA construct;
b) culturing the transformed tissue or cell on regeneration medium comprising copper at a concentration in a range from greater than 50 μM to less than or equal to 300 μM and a growth hormone at a concentration in a range from 0.05 to 10 mg l⁻¹ provided that the growth hormone is added to the regeneration medium if wheat tissue or cell has not been treated with a growth hormone after callus induction and prior to using the wheat regeneration medium; and
c) regenerating a transformed wheat plant from the transformed tissue or cell.

10. The method of claim 9 wherein the copper concentration is in a range from 100 μM up to less than or equal to 200 μM.

11. The method of claim 9 or 10 wherein the regeneration medium further comprises a selection agent.

12. The method of claim 11 wherein the selection agent is selected from the group consisting of kanamycin, geneticin, paromomycin, hygromycin, glyphosate, bialaphos, phosphinothricin, and sulfonylurea herbicide.

13. The method of claim 9 or 10 wherein the regeneration medium further comprises silver nitrate at a concentration in the range from 1 to 20 mg l⁻¹.

14. The method of claim 11 wherein the regeneration medium further comprises silver nitrate at a concentration in the range from 1 to 20 mg l⁻¹.

15. The method of claim 9 or 10 wherein the growth hormone is selected from the group consisting of 2,4-dichlorophenoxyacetic acid, indole-3-acetic acid, naphthaleneacetic acid, monochlorophenoxyacetic acid, 3,6-dichloro-o-anisic acid, 4-amino-3,5,6-trichloropicolinic acid, 6-benzylaminopurine, indole-3-butyric acid, 6-[4-hydroxy-3-methylbut-2-enylamino]purine, 6-furfurylaminopurine, 1-phenyl-3-(1,2,3-thiadiazol-5-yl)urea, abscisic acid, chlorocholine chloride and gibberellic acid.

16. The method of claim 11 wherein the growth hormone is selected from the group consisting of 2,4-dichlorophenoxyacetic acid, indole-3-acetic acid, naphthaleneacetic acid, monochlorophenoxyacetic acid, 3,6-dichloro-o-anisic acid, 4-amino-3,5,6-trichloropicolinic acid, 6-benzylaminopurine, indole-3-butyric acid, 6-[4-hydroxy-3-methylbut-2-enylamino]purine, 6-furfurylaminopurine, 1-phenyl-3-(1,2,3-thiadiazol-5-yl)urea, abscisic acid, chlorocholine chloride and gibberellic acid.
